(19) United States
(12) Patent Application Publication
(10) Pub. No.: US 2005/0106756 A1
(43) Pub. Date: May 19, 2005

(54) Microstructured separation device, and method for separating liquid components from a liquid containing particles

(76) Inventors: Gert Blankenstein, Dortmund (DE); Claus Marquordt, Dortmund (DE)

Correspondence Address: HOFFMAN, WASSON & GITLER, P. C.;Crystal Center 2, Suite 522, 2461 South Clark Street, Arlington, VA, 22202, US

(21) Appl. No.: 10/983,096

(22) Filed: Nov. 8, 2004

(30) Foreign Application Priority Data
Nov. 7, 2003 (DE) 103 52 535.1

(51) Int. Cl. G01N 33/543

(52) U.S. Cl. 436/523; 210/323.1

(57) ABSTRACT

A microstructured separation device for separating parts of a liquid which comprises liquid components and at least one type of particle and/or at least one complex of interconnected particles of at least one type, with the following features: the device has an inlet for the liquid, a collection section, and a transport path from the inlet to the collection section; the transport path includes, situated one after the other in the direction of transport, a resuspension section, an incubation section, a first separation section for holding back at least some of the complexes and/or for slowing down the movement of at least some of the complexes and/or at least some of the particles, and a second separation section for holding back at least some of the complexes and/or at least some of the particles and/or for slowing down the movement of the particles; and the first separation section and second separation section have a microstructure with one or more microstructure elements.

### BACKGROUND OF THE INVENTION

The present invention relates to a microstructured separation device for separating parts of a liquid which comprises liquid components and at least one type of particle and/or at least one complex of interconnected particles of at least one type.

Most biochemical tests are performed on cell-free blood fluids, blood plasma or blood serum, because the blood cells or their contents can distort the measurement results. In hematology, filtration and centrifugation techniques have hitherto mainly been employed for this separation. Complete separation of blood cells without destroying their membranes, and without releasing their content into the test solution, is achieved for example by means of a 15-minute centrifugation at 1500 revolutions per minute. Such a method is complex and time-consuming, for which reason alternative solutions have been sought. Moreover, the current procedures described here do not permit handling of small volumes in the range of a few microliters, which is of importance especially in the point-of-care area in clinical diagnostics and in pharmaceutical active substance research.

By contrast, extremely small sample quantities can be handled with the aid of microsystem technology. In addition, many analysis system components can be combined within a very small space. Medical diagnosis is in this way often made easier, less expensive, more patient-friendly and, above all, can be carried out closer to the patient.

A microstructured separation device for separating hematocrit from whole blood is known, for example, from document U.S. Pat. No. 6,319,719 B1. This document discloses a separation device having an inlet adjoined by a capillary transport path leading to a reaction region. A large number of obstacles are arranged along the transport path. To separate the hematocrit from a sample of the order of size of a drop of blood, the capillary transport path contains about 10⁵ obstacles. Each obstacle has a concave shape on its downstream side in relation to the direction of flow of the liquid. In the concave area of each obstacle there is a volume of 10⁻⁴ to 10⁻⁵ microliters in which hematocrit is selectively held back. The volume of all of the concave areas corresponds approximately to the volume of the hematocrit to be separated. The distance between the obstacles is, on the one hand, large enough to ensure that no filter effect is generated, and, on the other hand, the distance is small enough to minimize the volume of liquid contained in the capillary transport path. The smallest distance between two obstacles is preferably about 10⁻⁵ meters. The obstacles are preferably arranged as tight hexagons.

It is an object of the invention to propose a separation device with which the separation process can be performed rapidly. A further object of the invention is to propose a separation device with which liquid and particulate components can be separated from a liquid which contains certain particles. For example, it should be possible to separate some of the blood plasma and the white blood cells from the rest of the blood if only white blood cells and blood plasma are required for certain analysis purposes. It should further be possible, for example, for cellular blood components, bacteria or viruses to be separated from the other particles in the blood or to test the liquid which is depleted in blood plasma and in which the particles are present in accumulated form.

### SUMMARY OF THE INVENTION

A microstructured separation device has an inlet for the liquid, a collection section, and a transport path from the inlet to the collection section. In addition to having a first separation section for holding back the complexes and/or for slowing down the movement of the complexes and/or of at least some of the particles and a second separation section for holding back the complexes and/or at least some of the particles and/or for slowing down the movement of at least some of the particles, the transport path also has a resuspension section and an incubation section which is arranged before the first separation section in the direction of transport. Both the first separation section and also the second separation section have a microstructure with one or several microstructure elements. The microstructure elements are not necessarily configured in a specific manner. They need simply be configured such that the separation sections are able to fulfill the duties assigned to them. The incubation section provided in the separation device of the invention is intended to give substances added to the liquid the possibility of contributing to the formation of the complexes before the liquid reaches the first separation section.

At least one substance for producing the complexes from the particles and/or for promoting the production of the complexes from the particles can be arranged in the resuspension section of the transport path. It is thus not necessary for the substance to be added to the liquid before the separation device is filled. Instead, the liquid is introduced directly into the separation device and, after reaching the resuspension section, the liquid takes up the substance arranged in the resuspension section or, if various substances are arranged there, the substances.

In contrast to the test vessels customarily found in a normal laboratory, the ratio of the surface of the transport path to the enclosed volume of the transport path of the microstructured separation device is much increased. This results, inter alia, in the following deviations from the conditions found in macro technology.

Surface effects and capillary and adsorption phenomena often play a dominant role in relation to volume effects. At dimensions under 100 μm and flow velocities under 100 cm/sec, liquids flow in a laminar fashion, i.e. as a so-called stratified flow. No turbulence occurs. The liquids have low Reynolds numbers, typically below Re=100. In this way, mixing of liquids is not afforded through turbulence. On the other hand, because of the small dimensions, diffusion represents a rapid and efficient mixing mechanism.

The transport path is advantageously so configured that the liquid is moved by capillary force. In addition, liquids can be transported using other drive mechanisms such as electroosmotic force (EOF). The transport path of the device through which the liquid is to be transported is configured accordingly. This applies to its cross-sectional surfaces, cross-sectional configurations and surface properties.

Particles within the meaning of the invention can, for example, be solid particles of materials such as glass, plastics, resins, or particles of biological origin, such as prokaryotic and eukaryotic biological cells, cell agglomerates, cell fragments, organelles, macromolecules such as nucleic acids, proteins, etc., or a combination of solid particles and particles of biological origin, for example glass support coated with cells.

Complexes within the meaning of the invention are any accumulation of several interconnected particles in the liquid. These can be regularly arranged particles or randomly interconnected particles. The connection can be produced by forces acting between the particles. However, the connection can also be produced by an additional substance for connection of individual particles. The particles of a complex can be of identical type or of different types.

The complexes can in principle be generated by naturally occurring processes. According to the invention, however, they are formed, or their formation is accelerated, by the substance or several substances in the resuspension area being added to the liquid.

The separation device according to the invention satisfies the demands placed on it. In particular, it permits a much more rapid separation process. Complexes and/or for example large particles are initially held back or have their movement slowed down by the first separation section in such a way that the liquid components and individual particles not bound in complexes can pass rapidly into the second separation device in which the remaining particles that are not to be collected in the collection section are held back or slowed down. Only the liquid components and, possibly, some of the particles which are to be separated from the rest of the liquid are in the end collected in the collection section. Since, after complete filling of the collection area, no further liquid components with other particles contained in them or even complexes can enter into the collection area thus completely filled, it is possible to obtain rapid and reliable separation. Unlike the separation device known from the art, in a separation device according to the invention a small number of from 5 to 100 microstructure elements is sufficient for a successful separation process.

In the transport path, the separation device has the incubation section which, in the direction of transport, is arranged before the first separation section. A liquid which has taken up a substance in the resuspension section is transported into the incubation section, through which the liquid flows at such a speed that, during the dwell time of the liquid in the incubation section, the substance causes or accelerates the desired formation of the complexes. In this way, it is possible to ensure that, when the liquid reaches the first separation section, the complexes are formed or substantially formed. By means of the design of the incubation section (cross-sectional surface, length, surface properties such as roughness and wettability), the flow speed and thus also the dwell time of the liquid in the incubation section can be set in a reproducible manner.

The microstructure elements of the first separation section have pockets which are open in the direction of the inlet, i.e. counter to the direction of transport, and/or the microstructure elements at least in part enclose, with the boundary surfaces of the transport path adjacent to the microstructure elements, pockets which are open in the direction of the inlet.

A preferred device according to the invention permits separation of blood into plasma and hematocrit without addition of substances, by using the blood's inherent properties of forming cell aggregates, i.e. complexes. An example of complex formation that occurs under natural circumstances (without addition of a chemical) is erythrocyte aggregation, in particular rouleau formation, with slow-flowing or non-circulating blood. Here, the red blood cells (erythrocytes), measuring approximately eight thousandth of a millimeter (μm), arrange themselves in the shape of a roll of coins, partially branched, with the flat surfaces on one another, and form long chains. These can be visualized without too much difficulty using normal microscopy techniques (dark-field illumination or phase-contrast illumination) under a light-optical microscope with attached video camera. The structures of the device are configured to ensure that the flow speeds and, consequently, the shearing forces are so low that this rouleau formation can occur.

According to the invention, at least some of the microstructure elements of the first separation section can be columns or steles, which can have a circular, hexagonal, quadratic, rectangular or oval cross section. Moreover, at least some of the microstructure elements can have one or more webs. The web or webs can be arranged transversely or obliquely with respect to the direction of transport. The webs can also be bent or angled in a U-shape or V-shape so that they have open pockets counter to the direction of transport.

The microstructure elements of the first separation section advantageously delimit one or more first through-openings which have geometric dimensions allowing at least some of the particles and smaller complexes and also the liquid components of the liquid to pass through. Although the first through-openings thus configured allow some of the particles and/or at least some of the smaller complexes to pass through, they nevertheless slow down the transport of the particles and/or complexes, because these are temporarily held back on the microstructures or are able to pass through the first through-openings only after a deformation, for example in the case of red blood cells. Liquid components can pass unimpeded through the first through-openings. As a result of a collision of several such complexes, it is possible for larger complexes to form within one of the first through-openings.

The microstructure elements of the first separation section can also delimit first and/or second through-openings which have geometric dimensions allowing only the particles or certain types of particles and the liquid components to pass through. Complexes are held back by means of these first and/or second through-openings, whereas the particles or the certain types of particles are able to pass through, slowed down, or only after a deformation, which also slows down the transport of these particles. Liquid components can pass unimpeded through these first and/or second through-openings. Some of the second through-openings can be provided starting in or on the pockets of the microstructure elements of the first separation section. It is thus possible to ensure that, although some of the complexes are collected in the pockets and their further transport is impeded, some particles or some of the complexes and the liquid components can nevertheless flow onward through the second through-openings in the direction of transport. According to the invention, the first through-openings can have a width and/or height of 1 μm to 500 μm. The area of passage of the first through-openings can decrease in the direction of transport.

According to the invention, the width of the second through-openings can be from 1 μm to 500 μm, while the height can be from 0.1 μm to 100 μm. The area of passage of the second through-openings can, like the area of passage of the first through-openings, decrease in the direction of transport.

One possible way of arranging the substances in the resuspension section of a device according to the invention is for the substance or substances to adhere, dried on, to at least one boundary surface of the resuspension section. Another possibility is for at least one of the substances to be arranged in the form of a pellet, a tablet or a powder in the resuspension section. It is likewise possible for at least one of the substances to be mounted on a support, said support being arranged in the resuspension section. The substance or the support can in this case be arranged in a recess in one of the boundary surfaces of the resuspension section.

At least some of the particles can be of biological origin, for example cells or their organelles, viruses or similar. The creation of complexes from particles is generated, promoted or accelerated by means of one or more substances, by aggregation, agglutination and/or coagulation, etc.

In hematology, aggregation is understood as the reversible clumping-together of red blood cells by relative increase (fluid loss) or absolute increase of, above all, large proteins of the blood (agglomerins, e.g. fibrinogen, haptoglobin). Agglutination is understood as the in most cases irreversible bonding of antigen-carrying particles (erythrocytes, bacteria, or in passive indirect agglutination of latex particles, polystyrene particles) by suitable agglutinins such as antibodies or lectins. The antigen-antibody reaction causes clumping of particulate antigens. In direct agglutination, the agglutinating antibodies are directed against bacterial or cell-bound antigens; in indirect agglutination, soluble antigens are coupled to a solid carrier. The particles concerned are in most cases large enough to be visible by microscope.

To realize the invention, agglutinating substances used can also be agglutinating antibodies which agglutinate antigen-carrying particles located in the sample so that these form complexes. Such a reaction can be used, on the one hand, as an isolation method for removing certain particles from a particle-containing solution or, on the other hand, as an analysis method for detection of a certain particle in the sample solution. To this end, the separation section can be used simultaneously as a detection area. Particles that are difficult to identify visually are rendered visible by combining them into complexes and they are enriched (concentrated) in the separation area. They can thus be identified easily and conveniently by optical methods, for example by scatter or turbidity measurements, or by light-optical microscopy.

These antibodies counted among the substances within the meaning of the invention can also be applied to spherical supports. These spherical supports are often polymer or glass particles with a diameter of 0.05 μm to 100 μm.

The microstructure elements of the second separation section can comprise a stairway, spaced apart columns and/or one or more webs which, with a top part or cover of the device, form a gap or one of more through-openings. In principle, the second separation section can be designed in the manner described for example for the separation area of a separation device disclosed in the German patent application 10313201.5/44.

A further embodiment of the separation device according to the invention can have a branch section before the first separation section or between the first separation section and the second separation section, starting from which branch section a second transport path branches off from the first transport path. The branch section and the second transport path starting from the branch section ensure that, in the event of blockage of the first or second separation section through formation of a so-called “filter cake”, the liquid following on automatically flushes the first or second separation section. The particles or complexes deposited before the entrance or in the entrance area of the first or second separation section are carried off by the continuous flow of liquid into the second transport path. This ensures that the first or second separation section is always kept free for the separation process.

According to the invention, the length of the first transport path up to the second transport section is dimensioned in such a way that, because of the limited mobility of the complexes, the liquid components or liquid components and particles are first to reach the second separation section. In terms of its length, cross section, surface properties, and the design of the microstructure elements, the transport path can advantageously be configured such that only liquid components, possibly with certain particles, reach the collection area.

Because of the preferably hydrophilic properties of at least parts of the transport path, the more mobile liquid components of the sample fill the collection area more rapidly than do the particles or complexes which, on account of their mass, volume and size, are partially or completely held back in the separation area.

The method for separating parts of a liquid, the liquid components and at least one type of particle and/or at least one complex of interconnected particles of at least one type, has the following steps: After an incubation period during which at least individual complexes have formed, the liquid is applied to a first separation section of a separation device, for example of a separation device according to the invention. In the first separation section, the complexes are held back and/or the movement of the complexes and/or of certain types of particles or of all the particles is slowed down.

The separated parts of the liquid collected in the collection area can be analyzed in the collection section or they can be removed from the collection area for further analysis outside the separation device. The enriched particles and/or complexes can also be analyzed in the separation section itself or in the remaining part of the transport paths.

The complexes and/or the particles can be held back and/or the movement of certain types of particles or of all the particles can be slowed down in a second separation section of the separation device.

Prior to the start of the incubation period, at least one substance producing and/or promoting the production of complexes of interconnected particles can be added to the liquid. The liquid can be incubated in an incubation section of the separation device. It is also possible for the substance to be added to the liquid and resuspended from the liquid in a resuspension section of the separation device.

The complexes can be formed at least in part by agglomeration, agglutination and/or coagulation of the particles. The substance that can be added to the liquid can contain supports (parts) which are coated with antibodies or lectins and which, for example by an antigen-antibody reaction with surface antigens of the particles, can effect formation of agglutinates (complexes).

The mobility of the complexes and/or of the particles can be restricted at least in part by the microstructure elements in the first separation section, and the complexes can at least in part be held back by the microstructure elements of the first separation section.

According to the invention, the complexes can be held back by the microstructure elements of the second separation device and/or some of the particles can be held back by these microstructure elements and/or the movement of at least some of the particles can be slowed down by these microstructure elements.

According to the invention, the liquid or the separated parts of the liquid can be transported by capillary force and/or by another force of comparable magnitude.

According to the invention, the device can comprise a sample support in which the inlet, the transport path and the collection section are formed, and the device can comprise a top part or cover which advantageously covers the transport path and the collection section, i.e. the microstructured side of the sample support except for the inlet. The structured side of the sample support can be at least partially hydrophilized if the device is intended for samples with hydrophilic properties, e.g. aqueous samples or blood.

The methods according to the invention can also be applied to complex-forming liquids different than blood. Thus, certain particles can be complexed in a liquid, enriched (concentrated), and detected. For this purpose, the complexes enriched in the separation areas may also be of interest for analysis purposes, for example.

According to the invention, the agglutinating substances used can also be agglutinating antibodies which agglutinate antigen-carrying particles located in the liquid so that these form complexes. Such a reaction can be used, on the one hand, as an isolation method for removing certain particles from a particle-containing solution or, on the other hand, as an analysis method for detection of a certain particle in the sample solution. To this end, the separation section is used simultaneously as a detection area. Particles that are difficult to identify visually are rendered visible by combining them into complexes and they are enriched (concentrated) in the separation area, by which means they can be identified easily and conveniently by optical methods.

These substances can also be applied to spherical supports. These spherical supports are often polymer or glass particles with a diameter of 0.005 μm to 100 μm.

### BRIEF DESCRIPTION OF THE DRAWINGS

Illustrative embodiments for separation devices according to the invention are described in more detail with reference to the drawing, in which:

FIG. 1 shows a plan view of the lower part of a first separation device according to the invention;

FIG. 2 shows a cross section through the first separation device, along the line II-II in FIG. 1;

FIG. 3 shows a cross section through the first separation device, along the line III-III in FIG. 1;

FIG. 4 shows a plan view of part of a lower part of a second separation device according to the invention;

FIG. 5 shows a cross section through the second separation device, along the line V-V in FIG. 4;

FIG. 6 shows a cross section through the second separation device, along the line VI-VI in FIG. 4;

FIG. 7 shows a part of a third separation device according to the invention during a separation process;

FIG. 8 shows different examples of microstructure elements in a first separation section of a separation device according to the invention;

FIGS. 9 to 11 show a lower part of fourth, fifth and sixth illustrative embodiments, respectively, of a separation device according to the invention in plan view;

FIG. 12 shows a variant of the configuration of an inlet of one of the separation devices according to the invention;

FIG. 13 shows a plan view of the lower part of a seventh illustrative embodiment of a separation device according to the invention;

FIG. 14 shows a cross section through the seventh illustrative embodiment, along the line XIV-XIV;

FIG. 15 shows a cross section through the seventh illustrative embodiment, along the line XV-XV in FIG. 14;

FIG. 16 shows a plan view of the sample support of an eighth illustrative embodiment;

FIG. 17 shows a cross section through the eighth illustrative embodiment, along the line XVII-XVII in FIG. 16; and

FIG. 18 shows a plan view of the lower part, or sample support, of a simple device for carrying out the method according to the invention.

### DETAILED DESCRIPTION OF THE INVENTION

The illustrative embodiments shown in the figures for separation devices according to the invention show in some cases identical features and in some cases corresponding features, which are labeled with the same reference numbers.

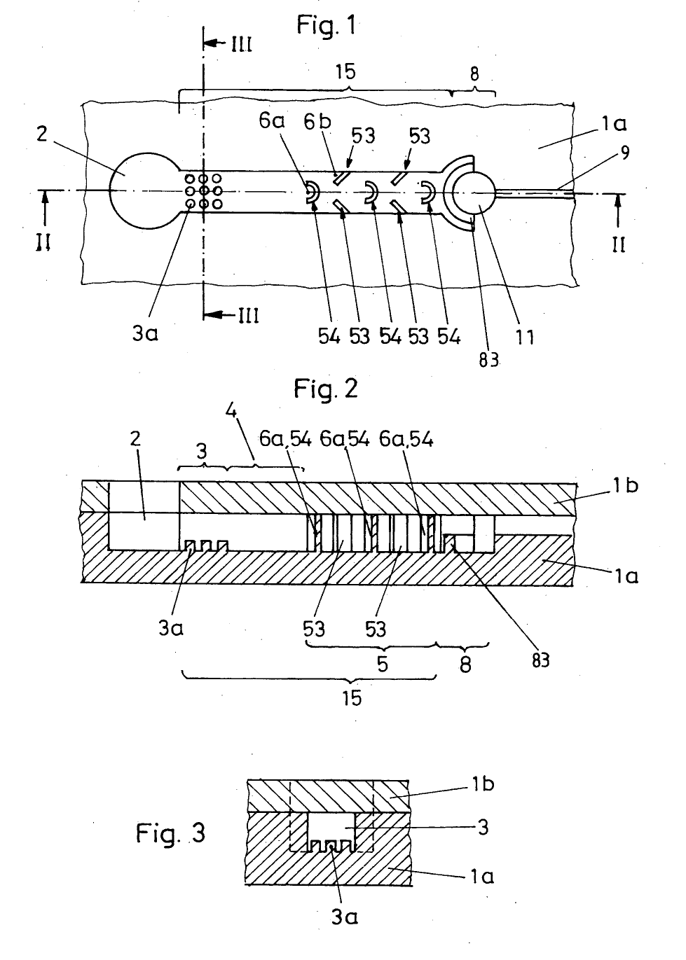

The first illustrative embodiment shown in FIGS. 1 to 3 has a lower part 1a in which an inlet 2 of circular cross section is formed. From this inlet 2, a first transport path 15 branches off laterally and extends as far as a collection section 11. The collection section 11 is connected to the environment via a vent channel 9. A capillary plug in the transport channel 9 can prevent escape of the liquid components collected in the collection section 11.

Such a capillary plug can be realized by means of abruptly changing geometric dimensions of the transport channel 9. Likewise, surface properties of the transport channel 9 can abruptly change, for example from hydrophilic to hydrophobic surfaces.

The transport path 15 between the inlet 2 and the collection section 11 is divided into different sections. Situated one after the other in the direction of transport of the liquid, that is to say in the direction from the inlet 2 to the collection section 11, there is a resuspension section 3, an incubation section 4, a first separation section 5 and a second separation section 8. Both the first separation section 5 and the second separation section 8 have a microstructure with microstructure elements 53, 54 in the first separation section 5 and microstructure elements 83 in the second separation section 8.

Besides the lower part 1a, which can be a support for a sample, the first separation device shown in cross section in FIGS. 1 to 3 has a cover 1b which covers the transport path 15, the collection section 11 and the vent channel 9 and leaves only the inlet 2 free.

The first separation device shown in FIGS. 1 to 3 is suitable for obtaining blood plasma from blood, where the hematocrit, during transport along the transport path 15, remains in the first separation section 5 and in the second separation section 8 of the transport path 15, so that only blood plasma collects in the collection section 11.

The hematocrit is separated from the blood plasma on account of the surface effects which arise during transport of the blood along the transport path 15.

The particle-containing liquid is separated in the first separation section 5 according to the following principle: The mobility of particles and complexes is so limited, compared to that of the rest of the liquid, that they are transported more slowly through a capillary than are the remaining components of the liquid. Through the formation of the complexes (bringing about an increase in mass, volume and size), the particles bound in complexes are less mobile than the unbound particles.

The movement and speed of the complexes in the continuous through-flow is further impaired by the microstructure elements 53, 54 arranged in the separation section 5 and preferably lying transversely with respect to the direction of flow. The particles thus bound in complexes are separated from the particle-containing sample liquid on account of the aforementioned change in mass, volume, charge and size.

Because of their viscoelastic properties, erythrocytes can also flow through gaps or capillaries which are smaller than their diameter or their thickness. The passage of an erythrocyte through capillary gaps with a gap opening of less than 5 μm is complex and is effected, after a delay, by a rolling movement which cannot be described according to the Hagen-Poiseuille law. If plasma is to be isolated from blood, the gap height between the web 83 of the second separation section 8 and the upper part of the device is preferably smaller than 5 μm, in order to hold back individual unbound blood cells from the sample liquid in the separation section 8. While the separation section 8 does not represent an obstacle, or represents only a minor obstacle, for plasma, the red blood cells are held up and slowed down in the separation section and can pass through the latter only slowly. Here, the depth of penetration of the red blood cells depends on the height of the gap and on the time till the collection chamber 11 is filled completely with liquid. The length of the separation sections 8 in the direction of flow is chosen such that the collection chamber 11 is already filled completely with the more mobile plasma before the first individual red blood cells have completely negotiated the separation section 8.

In the first separation section 5 in particular, the microstructure elements 53, 54 form pockets 6a, 6b which are open counter to the direction of transport, that is to say in the direction of the inlet 2. Seen in plan view, the microstructure elements 54 are designed as U-shaped webs whose two arms point in the direction of the inlet 2. The pocket 6a is formed between the U-shaped arms. The microstructure elements 53 of the first separation section 5 are designed as webs 53 which point obliquely in the direction of the inlet and which are connected to the lateral boundary surface of the transport path 15. The acute-angled area formed between the webs 53 and the lateral boundary surface of the transport path 15 forms the pocket 6b of these second microstructure elements 53. Complexes and particles of the blood (hematocrit) collect in the pockets 6a, 6b of the microstructure elements 54, 53 and their movement along the transport path is thus suppressed or at least slowed down, whereas smaller particles of the blood, although being slowed down, can still pass through the first separation section 5. These smaller particles, however, are held back in the second separation section 8 in which a web 83 checks or at least slows down the movement of the smaller particles of the hematocrit. Larger particles which, although being slowed down, still pass through the first separation section 5 of the transport path 15 are in any case held back by the microstructure element of the second separation section 8.

The separation device according to FIGS. 1 to 3 has a further feature, which concerns in particular the second embodiment of the invention as claimed in claim 3. Before the first separation section, the transport path 15 comprises the incubation section 4. In this section, or in incubation sections 4 in one of the other illustrative embodiments of separation devices according to the invention, substances added beforehand to the blood or to another liquid to be treated are able to act on the liquid. These substances are chosen such that they cause or at least accelerate the formation of complexes from particles contained in the liquid, for example the formation of complexes of red blood cells. Particularly in the case of blood, these complexes can be generated by aggregation, agglutination or coagulation of red and/or white blood cells or other cells or viruses contained in the blood.

The substances or substance causing or promoting the formation of complexes is received in a resuspension area 3 of the first separation device. While the liquid is flowing through the incubation section 4 in the direction of transport, the substance acts on the liquid so that the complexes are formed, or are substantially formed, when the liquid reaches the first separation section 5. Some of the complexes of the particles and blood cells are stopped by the microstructure elements 53, 54, while some are slowed down. The complexes which are stopped are collected in the pockets 6a, 6b of the microstructure elements, for example. The total volume of all the pockets preferably corresponds to the volume of the particles contained in the liquid, that is to say of the hematrocrit. At the end of the first separation section 5, a liquid is obtained which by and large only contains individual particles or blood cells that are not bound in complexes. These last particles or blood cells are stopped or slowed down in the second separation section 8 by the web 83 arranged there, so that only blood plasma without cellular components or the like arrives at the collection section, until said collection section is completely filled.

The substance 3a arranged in the resuspension section 3 can be applied, in tablet form, on the lower boundary surface of the transport path 15 or resuspension section 3.

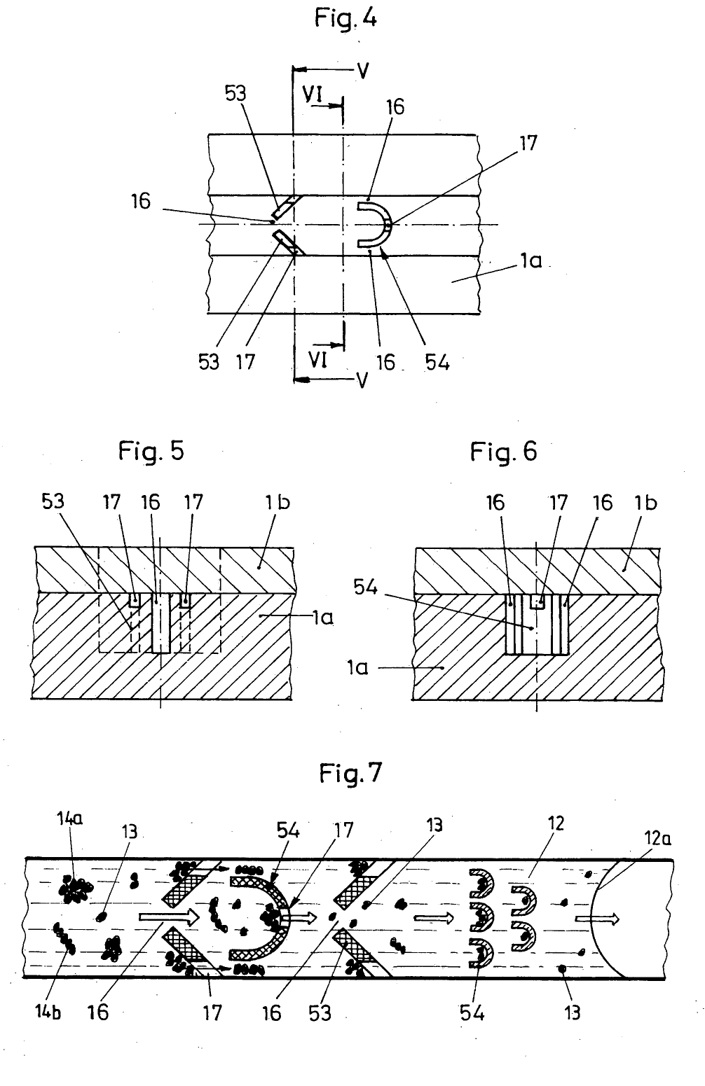

The cross section, shown in FIGS. 4, 5 and 6, of a first separation section of a second separation device shows a U-shaped web 54, and webs 53 arranged obliquely to the direction of transport, as microstructure elements which are similar to those in the first separation section 5 of the first illustrative embodiment of a separation device according to FIGS. 1 to 3. The first through-openings 16 are located between two mutually opposite webs, between a web 54 and the lateral boundary surface of the transport path 15, and between two adjacent webs 53, 53 or 54, 54 or 53, 54. The webs 53, 54 according to FIGS. 4 to 6 differ from the corresponding webs 53, 54 according to FIGS. 1 to 3 in that the webs are provided in the area of the pockets 6a and 6b with second through-openings 17. The second through-openings 17 have geometric dimensions which mean that only the smaller individual particles contained in the blood and/or the blood plasma can pass through, whereas complexes from the particles are held back. The second through-openings 17 are smaller than the first through-openings 16. The first through-openings 16 allow passage both to complexes of particles and to individual particles and also to the blood plasma. Through the second through-openings 17, air escapes from the pockets in the direction of the collection chamber 11, while liquid and particles or complexes of particles pass into the pockets.

FIG. 7 shows how blood plasma can be obtained from blood in the third separation device with the aid of the method according to the invention. To this end, FIG. 7 shows part of the transport path 15, namely the incubation section 4 and the first separation section 5. The figure also indicates the blood plasma 12 with a liquid front 12a, with individual cells 13, cell clusters 14a or so-called rouleaus 14b of blood cells swimming in the blood plasma. The cell clusters 14a and rouleaus 14b form in the incubation section under the influence of substances delivered to the resuspension section (not shown). The blood is transported from the incubation section 4 into the first separation section 5 by the capillary force acting along the transport path. In the process, cell clusters 14a, rouleaus 14b or individual cells 13 collect in the pockets 6a and 6b of the microstructure elements 53 and 54. The blood plasma 12 also flowing into the pockets 6a, 6b can pass through the second through-openings 17 and emerge again from the pockets in the direction of transport. The first through-openings 16 between two adjacent microstructure elements 53 and 54 and also between the microstructure elements 54 and the lateral boundary surfaces of the transport path 15 permit passage of individual cells 13 and also passage of complexes, for example the cell clusters 14a or rouleaus 14b. Because the complexes, formed by cell aggregates, or the individual cells are held back completely or partially, an area develops, at the front edge of the blood, which mainly contains blood plasma and only isolated cells. This mixture of blood plasma 12 and of individual cells 13 is transported by the transporting forces from the first separation section 5 into the second separation section 8 (not shown here).

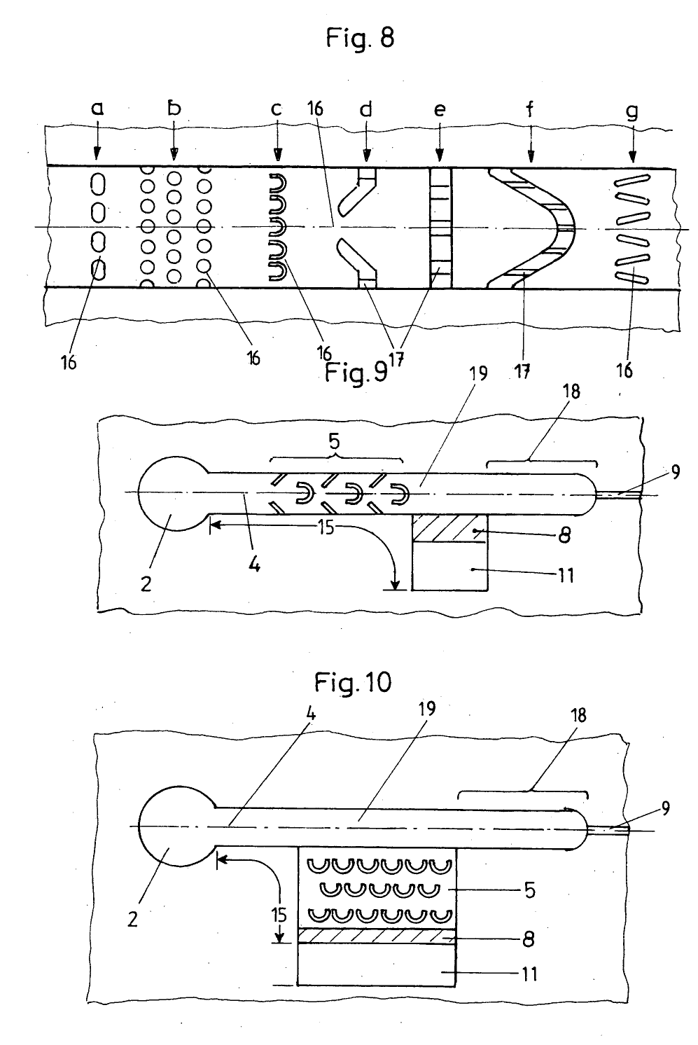

The variants of microstructure elements shown in FIG. 8 and used in the first separation section can be arranged alone or in a wide variety of combinations in a first separation area. The first microstructure elements a are webs or columns of substantially oval cross section which extend from a bottom boundary surface of the first separation section 5 to the top part of a separation device. The microstructure elements b are columns which are arranged behind one another in three rows. First through-openings 16 are located between two adjacent microstructure elements a, or between two adjacent microstructure elements b. The microstructure elements c are horseshoe-shaped webs which each delimit first through-openings 16 with adjacent horseshoe-shaped webs or with a lateral boundary surface of the first separation section 5. The webs can extend from the bottom boundary surface as far as the top part 1b of a separation device, or a gap can remain between the top of the horseshoe-shaped webs and the top part 1b. The latter also applies to the microstructure elements a and b, and to microstructure elements d which are angled webs extending counter to the direction of transport in the first separation section. Between the ends of two adjacent angled webs, there is a first through-opening 16.

The microstructure element e is a web extending across the full width of the transport path 15 from a first lateral boundary surface to a second lateral boundary surface. Second through-openings 17 are included in this web. A variant of the microstructure element e is formed by the microstructure element f, which is a single horseshoe-shaped web and, like the web e, contains second through-openings 17 and extends from a first lateral boundary surface to the second lateral boundary surface of the transport path. The microstructure elements g are webs which are arranged at an acute angle to the direction of transport in order to stop and/or slow down the complexes and to slow down individual particles, but allow liquid components to pass through largely unimpeded.

In addition to having the inlet 2 and the collection section 11, the fourth illustrative embodiment of a separation device according to the invention shown in FIG. 9 also has a transport path 15 comprising an incubation section 4, a first separation section 5, a second separation section 8 and, between the first separation section 5 and the second separation section 8, a branch section 19. The transport path 15 turns off from the branch section 19 at a right angle, while the branch section 19 is adjoined by a second transport path 18 which lies on a line with the incubation section 4 and the first separation section 5. A vent channel 9 leads to the outside from this second transport path 18.

In contrast to the previous examples, a volume flow is present, during the entire separation process, from the branch point in the direction toward the second transport path 18 and toward the second separation section 8. The liquid to be filtered flows parallel to the second separation section 8. Some of the liquid is accordingly drawn off transversely in the direction to the collection section 11. Because of the continuous flow of the liquid to be separated at the branch section 19, particles are carried with the volume flow into the second transport path 18, and the coverage of surface of the second separation section 8 is reduced. The degree of coverage can be varied as a function of the volume flow. However, the volume flow is always laminar, with Reynolds numbers of less than 100. In illustrative embodiments in which the liquid is driven exclusively by capillary force, the volume flow can be set via the dimensions of the channel and the surface properties.

FIG. 10 shows the fifth separation device according to the invention, which is very similar to the fourth illustrative embodiment according to FIG. 9. In contrast to the fourth illustrative embodiment according to FIG. 9, however, the branch section 19 in the fifth illustrative embodiment according to FIG. 10 is arranged before the first separation section. Both the first separation section 5 and also the second separation section 8 are arranged parallel to the branch section 19 and parallel to the direction of transport of the liquid from the branch section 19 to the second transport path 18. The second transport path 18 is arranged in a linear extension of the incubation section 4. By arranging the first separation section 5 parallel to the branch section 19, the inlet area of the first separation section 5 is automatically flushed. The inlet area of the first separation section 5 is not blocked by a “filter cake” formed there. A constant volume flow is obtained in the first transport path 15 and in the second transport path 18 until filling of the collection section 11 is complete.

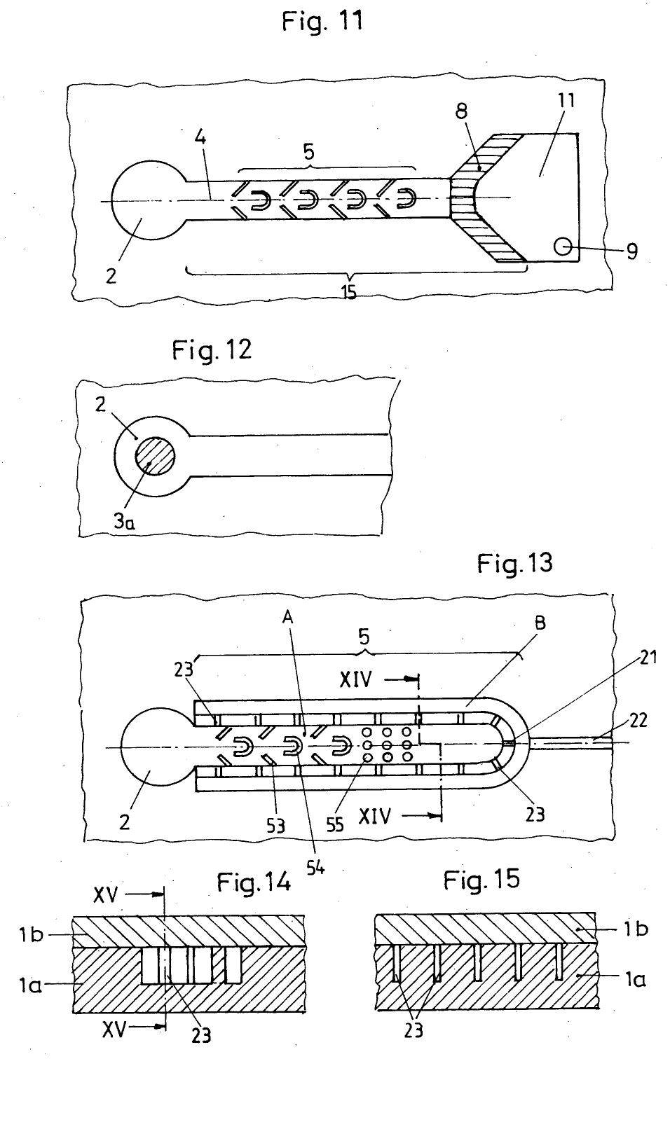

The sixth illustrative embodiment of a separation device according to the invention shown in FIG. 11 is similar to the illustrative embodiment according to FIG. 1, but, in contrast to the first illustrative embodiment according to FIG. 1, no resuspension section 3 is provided in the transport path 15. A web (for example like the microstructure element e or f in FIG. 8) with slits running perpendicular to the bottom boundary surface is provided as the microstructure element of the second separation section 8. Behind this second separation section 8, the liquid components of the liquid collect in a collection section 11. From the collection section 11, a vent channel 9 is routed through a bottom boundary surface of the collection section 11.

FIG. 12 shows a detail of a separation device according to the invention. In the inlet 2, the resuspension section 3a is provided as a circular surface on the bottom boundary surface of the inlet 2. The liquid introduced into the inlet 2 directly encounters the substance of the resuspension section 3a, by which means chemical or biochemical reactions between the substance and the particles in the liquid are triggered or accelerated, in order to generate, from the particles, complexes containing identical or different particles.

The seventh illustrative embodiment of a separation device according to the invention, shown in FIGS. 13, 14 and 15, likewise has an inlet 2, a transport path 15, and a collection section, but the second separation section and the collection section are not shown. FIG. 13 shows only the inlet and the first separation section 5. This first separation section 5 has a web which is U-shaped when seen in plan view and which extends from the bottom boundary surface of the first separation section 5 to the top part 1b of the separation device. This web separates two areas of the separation device which, in flow technology terms, lie one behind the other in the direction of flow. A first area A is arranged between the arms of the web and is connected directly to the inlet 2. The second area B is formed by a collection channel which surrounds the outside of the U-shaped web and is connected via a transport channel 22 to the second separation section (not shown). The first area of the first separation section 5 is provided with microstructure elements, such as, for example, webs 53 arranged obliquely with respect to the direction of transport, U-shaped webs 54 or columns 55 which, in the manner already described, prevent or slow down the transport of complexes and slow down the transport of individual particles. At the end of the first area A in the direction of flow, the U-shaped web has a through-opening 21 in its arch. The arch and the arms of the U-shaped web have, at regular intervals, slits extending from the bottom boundary surface of the first separation section 5 to the top part 1b of the separation device. The width of the slits 23 is dimensioned so that individual particles and liquid components of the liquid can pass through. The slits 23 form second through-openings within the meaning of the invention.

The separation device according to the invention shown in FIGS. 13 to 15 functions in the following way. The liquid is introduced into the inlet 2, from where it is transported by capillary force from the start to the end of the first area A of the first separation section 5. In this process, individual complexes and/or particles are stopped or slowed down by the microstructure elements 53, 54, 55 in the first area A. The liquid components and individual particles pass into the slits 23 in the U-shaped web. Since the cross-over from the slits 23 to the second area B of the separation section 5 represents a capillary stop for the liquid components, the liquid is initially not transported through the slits 23 into the second area B. The through-opening 21 provided in the arch is not configured as a capillary stop, and the liquid entering this through-opening 21 is able to pass into the second area B unimpeded. This can be achieved, for example, by notching or similar. As soon as the liquid components have completely filled the first area A and the through-opening 21 in the arch of the U-shaped web is wetted, liquid passes through the through-opening 21 into the second area B and fills the latter. The liquid wets the outside of the U-shaped web and the slits 23, by which means the capillary stop on the outside of the U-shaped web is annulled. The liquid lying in the slits 23 can then emerge from said slits 23, and the liquid and the individual particles contained in it then begin to be transported from the first area A into the second area B of the first separation section 5. Liquid components of the liquid collect with individual particles in the second area B. Because of the capillary force acting in the second area B and in the transport channel 22, this mixture of liquid components and individual particles is transported to a second separation section in which the individual particles are removed from the liquid in the manner already described several times above.

In an alternative design of the separation device according to FIGS. 13 to 15, the U-shaped web with its microstructure elements (slits) can be configured such that said U-shaped web already forms the second separation section of the separation device according to the invention, and such that the second area outside the U-shaped web serves as the collection section of the separation device according to the invention.

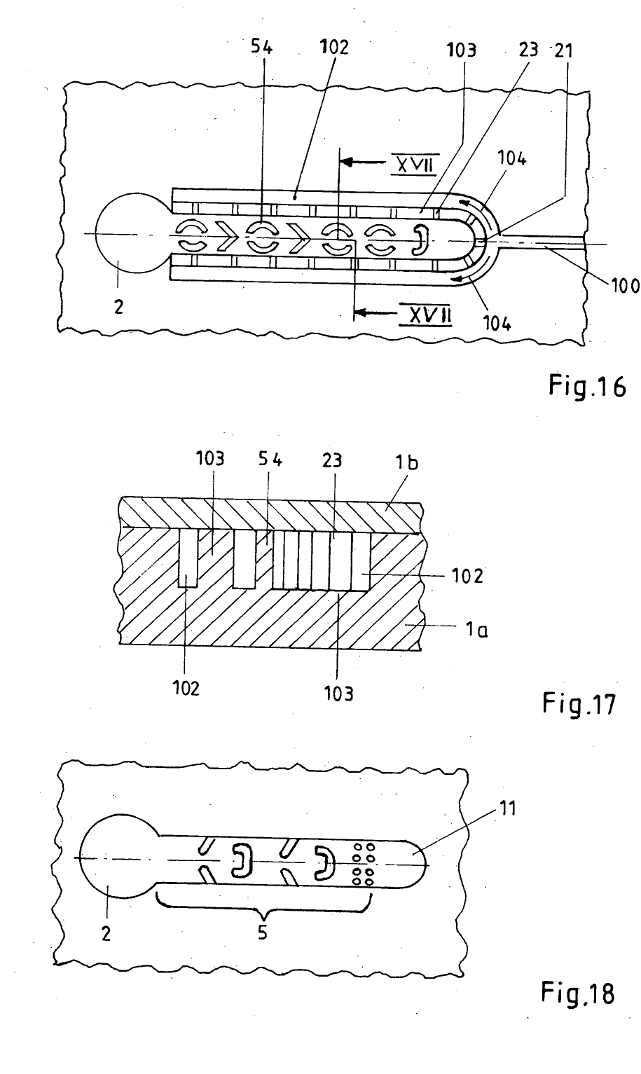

The device in FIGS. 16 and 17 has a design similar to the device shown in FIGS. 13 to 15. The first separation section is enclosed completely by a web 103 and also comprises this web 103. The web 103 is interrupted by a multiplicity of slits 12 which connect the first separation section to a channel 102 which is arranged parallel to the first separation section and is designed as a collection area. Here, the slits 23 preferably have the same depth as the collection channel 102. The slits form a capillary stop for the liquid in the direction of transport from the first separation section to the collection channel 102. The slits 23 have a depth of 1 μm to 100 μm, a width of 1 mm to 500 μm, and a length of at least 50 μm. The through-opening 21 has the same depth as the collection channel 102 and does not represent a capillary stop for the liquid.

After the particle-containing liquid has been introduced into the inlet area, the separation section is completely filled by means of capillary force. Particles and complexes are partially held back by the microstructure elements 53- and 54. As soon as the liquid has filled the through-opening 21, the liquid flows into the collection channel 102 and fills this in the direction of the inlet opening (see arrow 104). The cross section of the collection channel 102 is smaller than the cross section of the collection channel 100, by which means the liquid passing through the through-opening 21 preferably first fills the collection area 102 and only then flows onward through the collection channel 100. During the process of filling of the collection area 102, the individual slits 23 are wetted, by which means their capillary stop function is annulled, with the result that the practically stationary liquid present inside the web 103 can flow through the individual slits 23 in the direction of the collection channel 100. In this process, some particles or complexes are held back in the microstructures 54, now lying in the direction of flow, and by the slits 23, so that a solution largely depleted in particles flows through the collection area 102 in the direction of the collection channel 100, which leads to the second separation section (not shown).

The sample support shown in FIG. 18, like the other devices shown in the previous figures, is suitable for carrying out the method according to the invention as claimed in claim 30. For this purpose, the sample support has, besides the inlet 2, only the first separation area 5, with microstructure elements, and the collection area 11.

1. A microstructured separation device for separating parts of a liquid which comprises liquid components and at least one type of particle and/or at least one complex of interconnected particles of at least one type.
the device comprises an inlet for the liquid, a collection section, and a transport path from the inlet to the collection section;
the transport path comprises, situated one after the other in the direction of transport, a resuspension section, an incubation section, a first separation section for holding back at least some of the complexes and/or for slowing down the movement of at least some of the complexes and/or at least some of the particles, and a second separation section for holding back at least some of the complexes and/or at least some of the particles and/or for slowing down the movement of the particles;
the first separation section and second separation section have a microstructure with one or more microstructure elements.

2. A microstructured separation device, wherein microstructure elements of a first separation section have pockets open in a direction of an inlet and/or the microstructure elements at least in part enclose, together with boundary surfaces of a transport path adjacent to the microstructure elements, pockets open in the direction of the inlet.

3. The separation device as claimed in claim 1, wherein the microstructure elements of the first separation section comprise columns or steles.

4. The separation device as claimed in claim 3, wherein the columns have a rectangular or oval cross section.

5. The separation device as claimed in claim 2, wherein the microstructure elements of the first separation section comprise at least one web.

6. The separation device as claimed in claim 5, wherein the webs are arranged transversely or obliquely with respect to the direction of transport.

7. The separation device as claimed in claim 1, wherein the microstructure elements of the first and second separation sections delimit one or more first through-openings which have geometric dimensions allowing the particles and the complexes to pass through.

8. The separation device as claimed claim 1, wherein the microstructure elements of the first separation section delimit first and/or second through-openings which have geometric dimensions allowing only the particles or particles of certain types to pass through.

9. The separation device as claimed in claim 8, wherein the second through-openings are provided in part in the pockets of the microstructure elements of the first separation section.

10. The separation device as claimed claim 7, wherein the first through-openings have a width and/or height of 1 μm to 500 μm.

11. The separation device as claimed in claim 7, wherein an area of passage of the first through-openings decreases in the direction of transport.

12. The separation device as claimed in claim 8, wherein the second through-openings have a width of 1 μm to 500 μm and/or a height of 0.1 μm to 10 μm.

13. The separation device as claimed claim 8, wherein an area of passage of the second through-openings decreases in the direction of transport.

14. The separation device as claimed in claim 1, wherein at least one substance for producing the complexes from the particles and/or for promoting the production of the complexes from the particles is arranged in the resuspension section of the transport path.

15. The separation device as claimed in claim 14, wherein the at least one substance adheres, dried on, to at least one boundary surface of the resuspension section.

16. The separation device as claimed in claim 14, wherein at least one of the substances is arranged in the form of a pellet, a tablet or a powder in the resuspension section.

17. The separation device as claimed claim 14, wherein at least one of the substances is mounted on a support, or the support is immersed in the substance, said support being arranged in the resuspension section.

18. The separation device as claimed in claim 14, wherein at least some of the particles are of biological origin, cells or their organelles, viruses, and the substance or one of the substances causes, promotes or accelerates an aggregation, agglomeration, agglutination and/or coagulation of the living particles.

19. The separation device as claimed in claim 18, wherein the substance or one of the substances at least partly binds to an antigen fraction on the surface of the cell.

20. The separation device as claimed claim 14, wherein the substance or the support is arranged in a recess in one of the boundary surfaces of the resuspension section.

21. The separation device as claimed in claim 1, wherein the microstructure elements of the second separation section comprise a stairway.

22. The separation device as claimed claim 1, wherein the microstructure elements of the second separation section comprise columns spaced apart from one another.

23. The separation device as claimed claim 1, wherein the microstructure elements of the second separation section comprise one or more webs.

24. The separation device as claimed claim 1, wherein the separation device has a branch section before the first separation section or between the first separation section and the second separation section, from which branch section a second transport path branches off from the first transport path.

25. The separation device as claimed in claim 1, wherein the length of the transport path up to the collection section is dimensioned in such a way that, because of the chromatographic effect, only liquid components or liquid components and particles of selected types reach the collection section.

26. The separation device as claimed claim 1, wherein the length of the transport path up to the second separation section is dimensioned in such a way that, because of the chromatographic effect, only liquid components, or liquid components and particles, reach the second separation section.

27. The device as claimed in claim 1, wherein the incubation section has a length, a cross section, a configuration and/or surface properties by which the incubation time is set.

28. A method for separating parts of a liquid using the separation device of claim 1 which comprises liquid components and at least one type of particle and/or at least one complex of interconnected particles of at least one type, with the following steps:
after an incubation period during which at least individual complexes have formed, the liquid is applied to a first separation section of the separation device;
in the first separation section, the complexes are held back and/or the movement of the complexes and/or of certain types of particles is slowed down by microstructure elements;
the separated parts of the liquid components and of the particles and/or complexes are then collected in a collection section of the separation device.

29. The method as claimed in claim 28, wherein the complexes and/or the particles are held back and/or the movement of certain types of particles is slowed down in a second separation section of the separation device.

30. The method as claimed in claim 28, wherein, prior to the start of the incubation period, at least one substance producing and/or promoting the production of complexes of interconnected particles is added to the liquid.

31. The method as claimed in claim 30, wherein the substance, the supports or the particles are polymer spheres or glass spheres with a diameter of 0.05 μm to 200 μm.

32. The method as claimed in claim 31, wherein the supports are coated with one or more substances.

33. The method as claimed in claim 28, wherein the liquid is introduced into an incubation section of the separation device for incubation.

34. The method as claimed in claim 33, wherein the at least one substance is resuspended in a resuspension section of the separation device.

35. The method as claimed in claim 28, wherein the complexes are formed at least in part by agglomeration, agglutination and/or coagulation of the particles.

36. The method as claimed in claim 28, wherein the substance comprises antibody-coated parts, and the complexes are formed at least in part by binding of antigen fractions on membranes of the biological cells included in the particles.

37. The method as claimed claim 28, wherein the movement of the complexes and/or of the particles is slowed down at least in part by the microstructure elements in the first separation section, and the complexes are at least in part held back by said microstructure elements.

38. The method as claimed in claim 28, wherein the complexes are held back by the microstructure elements of the second separation device and/or some of the particles are held back by these microstructure elements and/or the movement of at least some of the particles is slowed down by these microstructure elements.

39. The method as claimed claim 28, wherein the separated parts of the liquid components and of the particles and/or complexes are analyzed in the collection section.

40. The method as claimed in claim 28, wherein the particles and/or complexes concentrated in the first separation section or in the second separation section are analyzed.

41. The method as claimed in claim 28, wherein particles and/or complexes are analyzed in the transport paths.

42. The method as claimed in claim 28, wherein the analysis comprises optical and/or electrochemical detection.

43. The method as claimed in claim 28, wherein, in a second collection section, a type of particle is concentrated which is different than that in the first collection section.

44. The method as claimed in claim 28, wherein the collection sections contain reagents.

45. The method as claimed in claim 28, wherein the liquid is transported by capillary force or by forces of comparable order, for example electroosmotic force.
